(19) United States
(12) Patent Application Publication
(10) Pub. No.: US 2004/0258719 A1
(43) Pub. Date: Dec. 23, 2004

(54) External preparation for treating dermatosis and pruritus due to hemodialysis

(76) Inventor: Yuichiro Takahashi, Yamato-shi (JP)

Correspondence Address: BIRCH STEWART KOLASCH & BIRCH, PO BOX 747, FALLS CHURCH, VA, 22040-0747, US

(21) Appl. No.: 10/464,549

(22) Filed: Jun. 19, 2003

(51) Int. Cl. A61K 31/60; A61K 7/00

(52) U.S. Cl. 424/401; 514/165

(57) ABSTRACT

Method for treating dermatosis and pruritus due to hemodialysis by applying to a patient suffering from said diseases due to hemodialysis the external preparation containing Aspirin as an active ingredient.

### BACKGROUND OF THE INVENTION

1. Field of the Invention

The present invention relates to therapeutic agents for xerosis cutis (skin-dry), dermatosis such as exanthema, or pruritus which becomes problem to patients receiving hemodialysis treatment.

In more detail the present invention relates to external preparations for treating dermatosis and/or pruritus due to hemodialysis, which contains acetylsalicylic acid as an active ingredient, and a method for treating said diseases by administering transdermally said external preparations in an amount of an effective dose to patients suffering from said diseases.

2. Description of the Related Art

Recently, with increase of patients suffering from endocrinism such as diabetes, and dysmetabolism such as nephropathy, patients who receive hemodialysis treatment are increasing. In the patients receiving hemodialysis treatment, turning for the deterioration of the skin condition and strong itching on body have become severe problem.

In regard to itching or pain on the patient receiving hemodialysis treatment, many studies have been done, but the mechanism thereof has not dissolved and that said patients are hardly cured comparing with the patients receiving no hemodialysis treatment. In addition there is none of agents effective to pruritus during hemodialysis treatment.

Nowadays many antipruritic agents for oral administration such as antihistamines etc. are sold. In case of such an agent being taken, it is anxious for its side effects, such as sleepiness, worthlessness feeling, etc. It is more difficult to use an antipruritic agent for oral administration to the patient receiving hemodialysis treatment, because frequencies of the side effects to the patients are higher comparing with ones to healthy persons.

On the other hand, an activity to dermatosis and an antipruritic activity of an external preparation containing an antihistamine or a nonsteroidal antiinflammatory agent are not satisfactory. Especially the preparation containing an antihistamine is also anxious for its side effects such as dermal anaphylaxis, and the preparation containing a nonsteroidal antiinflammatory agent is also anxious for its side effects, such as dermal irritation, contact dermatitis, etc.

Furthermore, although steroids for an external application are very useful for treatment of eczema, skin pruritus, etc., these steroids are not only anxious for their side effects, such as atrophia cutis, steroid flush, angiotelectasis, etc., when repeatedly taken, but also these steroids are transdermally absorbed to migrate to blood and have a possibility to give systemically bad effects.

Acetylsalicylic acid (Hereinafter it may be written as Aspirin.) has a strong analgesic activity, an antifebrile activity and an antirheumatic activity being less in its side effects and being superior in its safety. Therefore, acetylsalicylic acid has been widely used from of old.

Recently studies for new uses of external preparations containing acetylsalicylic acid have been done. For example, ointments for treating neuralgia (Japanese Patent Pub. A3-72426), external preparations for treating skin injury (Japanese Patent Pub. A9-235232), a transdermal administration system for treatment of thrombosis and for prophylactic treatment of cancer (Japanese Patent Pub. Tokuhyo 8-504198) are illustrated.

However, any external preparation containing acetylsalicylic acid for treating deterioration of skin condition and pruritus on body for the patients receiving hemodialysis treatment, and therapeutic effects thereof have not been reported.

### SUMMARY OF THE INVENTION

An object of the present invention is to provide external preparations which have an activity for improvement of skin condition and an excellent antipruritic activity on the patients receiving hemodialysis treatment and are less in their side effects.

The present invention relates to external preparations for treating dermatosis and/or pruritus due to hemodialysis, which contains acetylsalicylic acid as an active ingredient and a method for treating said diseases by administering transdermally said external preparations in an amount of an effective dose to patients suffering from said diseases.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a photo of a knee of a patient receiving hemodialysis treatment before administration of the external preparation of the present invention.

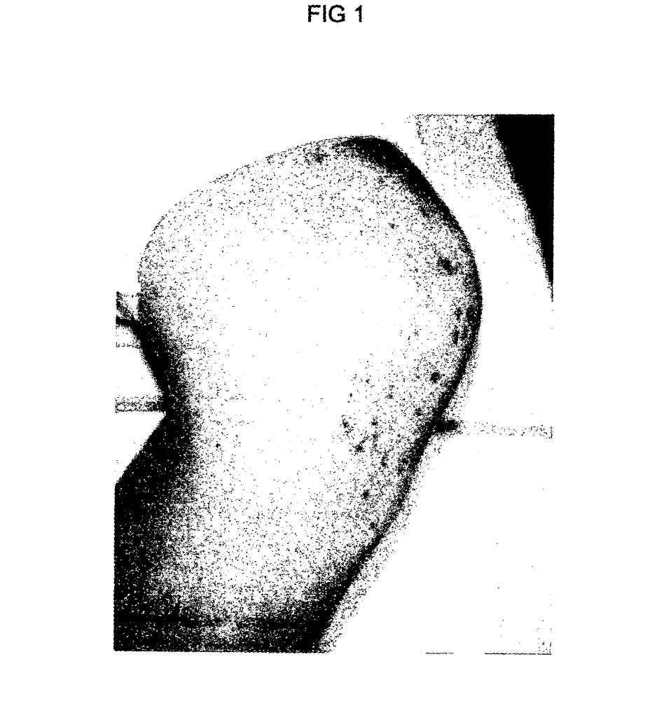

FIG. 2 is a photo of the same lesion of the patient after administration of the external preparation of the present invention.

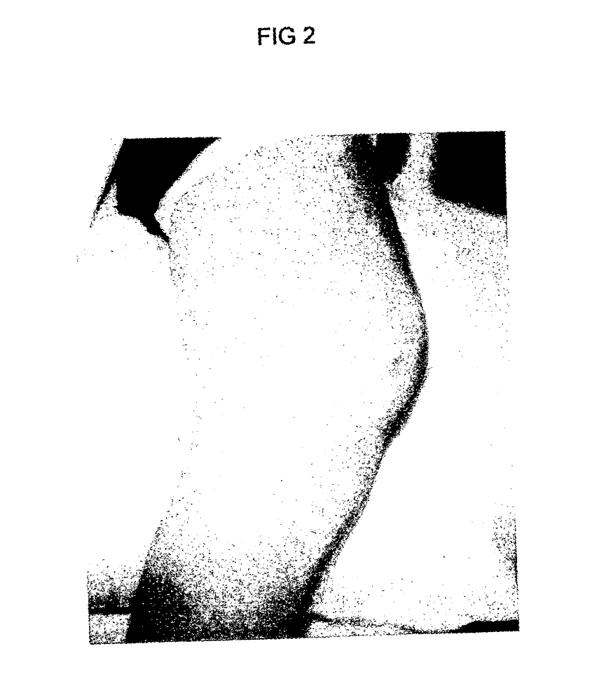

### DETAILED DESCRIPTION

The present inventors have earnestly studied and as a result, have found that when an external preparation containing acetylsalicylic acid as an active ingredient is administered to a patient receiving hemodialysis treatment, the preparation is less in its side effects and shows an excellent antipruritic activity and an excellent activity to dermatosis. Thus the present invention has been completed.

Namely, the present inventors have prepared the external preparation containing acetylsalicylic acid and when the preparation has been applied to, for example a lesion with itching and dermatosis, such as injured skin, eczema, ruber, ulticaria, prutitus, bulla, edema, etc., the excellent therapeutic activities thereto have been found.

Acetylsalicylic acid contained in the external preparation of the present invention is described in the Pharmacopoeia of Japan XIII and USP 26.

The dosage of Aspirin transdermally administered to a patient depends on the condition of the patient, but it is usually 0.01-200 mg/day/body weight (60 kg).

The external preparation of the present invention is applied to in the usual manner, such as, applied directly to the affected legion or is once painted or immersed on the cloth and then applied to the affected legion.

The diseases which the external preparation of the present invention is applied to are xerosis cutis, with itching and dermatosis, such as injured skin, eczema, rubor, ulticaria, pruritus, bulla, edema, etc. in patients receiving hemodialysis treatment.

The amount of acetylsalicylic acid contained in the external preparation of the present invention depends on form of the preparation, but is 0.05-80%, preferably 0.05-70%, more preferably 0.1-50% per total amount by weight. When the amount of acetylsalicylic acid is more than 80% by weight, it is impossible to maintain the physical property as an external preparation. When the amount of acetylsalicylic acid is less than 0.05% by weight, the antipruritic activity by acetylsalicylic acid does not show enough. The amount as more than 80% or less than 0.05% by weight, therefore is not preferable.

The external preparation of the present invention is not limited as far as it is the preparation in which acetylsalicylic acid can be directly applied on the local surface of skin, such as ointments, solutions (e.g. suspensions, emulsions, lotions), cataplasms, patches, plasters, tapes, aerosols and external powders (powders for external use).

As other ingredients except acetylsalicylic acid of the external preparation of the present invention can be used any ingredient used in the ordinarily external preparations.

In case of ointments, creams, gels and lotions, bases, such as white vaseline (petrolatum), yellow vaseline, lanolin, purified bee wax, cetanol, stearyl alcohol, stearic acid, hydrogenated oil, hydrocarbon gel, polyethylene glycol, liquid paraffin and squalane; solvents or solubilizing agents, such as oleic acid, isopropyl myristate, glycerol triisooctanoate, crotamiton, diethyl sebacate, diisopropyl sebacate, diisopropyl adipate, hexyl laulate, a fatty acid, a fatty acid ester, an aliphatic alcohol, and a plant oil; antioxidants, such as a tocopherol derivative, L-ascorbic acid, dibutylhydroxytoluene and butylhydroxyanisole; antiseptics such as p-hydroxybenzoate; humectants, such as glycerin, propylene glycol and sodium hyaluronate; surfactants, such as a polyoxyethylene derivative, a glycerol fatty acid ester, a sucrose fatty acid ester, a sorbitan fatty acid ester, a propylene glycol fatty acid ester and lecithin; thickening agents, such as carboxyvinyl polymer, xanthan gum, carboxymethyl cellulose, sodium carboxymethyl cellulose, hydroxypropyl cellulose and hydroxypropylmethyl cellulose; stabilizers; preservatives; absorption enhancers; and other suitable fillers may be added.

In case of tapes, tacking agents, such as a stylene-isoprene-stylene block copolymer and an acrylate resin; tackifier resins, such as an alicyclic saturated hydrocarbon resin, a hydrogenated rosin resin and a terpene resin; softeners, such as liquid gum and liquid paraffin; antioxidants such as dibutylhydroxytoluene; polyhydric alcohols such as polyethylene glycol; absorption enhancers such as oleic acid; surfactants such as a polyoxyethylene derivative; and other suitable fillers may be added. In addition a water-absorbable polymer, such as sodium polyacrylate and polyvinyl alcohol, and a small amount of purified water may be added to prepare tape preparations containing water.

In case of aerosols, bases, such as white vaseline (petrolatum), yellow vaseline, lanolin, purified bee wax, cetanol, stearyl alcohol, stearic acid, hydrogenated oil, hydrocarbon gel, polyethylene glycol, liquid paraffin and squalane; solvents or solubilizing agents, such as oleic acid, isopropyl myristate, isopropyl adipate, diisopropyl sebacate, glycerol triisooctanoate, crotamiton, diethyl sebacate, hexyl laurate, a fatty acid, a fatty acid ester, an aliphatic alcohol and a plant oil; antioxidants, such as a tocopherol derivative, L-ascorbic acid, dibutylhydroxytoluene and butylhydroxyanisole; antiseptics such as p-hydroxybenzoate; humectants, such as glycerin, propylene glycol and sodium hyaluronate; surfactants, such as a polyoxyethylene derivative, a glycerol fatty acid ester, a sucrose fatty acid ester, a sorbitan fatty acid ester, a propylene glycol fatty acid ester and lecithin; thickening agents, such as carboxyvinyl polymer, xanthan gum, carboxymethyl cellulose, sodium carboxymethyl cellulose, hydroxypropyl cellulose and hydroxypropylmethyl cellulose, as used in the ointments, the creams, the gels, the suspensions, the emulsifying agents or the lotions; stabilizers; buffering agents; sweetening agents; suspending agents; emulsifying agents; flavors; preservatives; solubilizing agents; and other suitable fillers, may be added.

In case of external powders, fillers, such as potato starch, rice starch, corn starch, talc and zinc oxide, and other suitable additives may be added.

The external preparation of the present invention can be prepared, for example by well kneading each ingredient, if necessary with a suitable base, in accordance with a usual manner.

In case of preparing ointments, using fat, fatty oil, lanolin, wax, resin, plastic, glycols, higher molecular weight alcohols, glycerin, water, emulsifier, suspending agent, or other suitable ingredients as a starting material, or these ingredients as a base and adding a medicament thereto the resulting mixture are kneaded to become homogeneity. Namely, the ingredients for the base are dissolved under heating and mixed to become homogeneity and if necessary, absorption enhancer, antioxidant, preservative, purified water, etc., is added. Then acetylsalicylic acid in powders is added under stirring and kneaded to prepare ointments or creams.

For example, in case of preparing oleaginous ointments, a base is melted under warming and mixed and after the mixture is semi-cooled, a medicament in powder or solution is mixed with a part of the base. Then the mixture is mixed with the rest of the base to become homogeneity.

For example, in case of preparing emulsified or water-soluble ointments, after the solid base is melted on a water bath and to the melted base keeping at about 75° C. is added the water-soluble base which is dissolved in water kept at the same temperature or a little higher. The mixture is kneaded to become homogeneity. In case of adding another medicament to this mixture, according to the property of the base, a water-soluble or oil-soluble medicament is first mixed with a part of the base and then homogeneously with the rest of the base.

In case of preparing tapes, to a tacking agent such as an acrylate resin or a stylene-isoprene-stylene block copolymer are added tackifier resins, such as an alicyclic saturated hydrocarbon resin, a hydrogenated rosin resin and a terpene resin, softeners, such as liquid gum and liquid paraffin, absorption enhancers and antioxidants. The mixture is dissolved in an organic solvent such as toluene, or is melted under heating and the mixture is well mixed. To the mixture is mixed a medicament in powder or in solution. The mixture is spread on a release paper. In case of a soluble type, after spreading and dry, the paper is laminated on a flexible support such as with polyurethane film, polyethylene film, poly chlorinated vinyl film, woven cloth or unwoven cloth and then cut in a desired size.

In case of preparing lotions, a medicament, a solvent, an emulsifier, a suspending agent, etc., are added to an aqueous solution and the mixture is homogenized. In case of preparing suspending lotions, after pulverizing the medicament and making it wettable with glycerin or ethanol etc., then thereto is gradually added a solution of a suspending agent or a base of the lotion and the mixture is homogenized. In case of preparing emulsifying lotions, an oil-soluble medicament and an oil layer are put in one vessel, and an aqueous layer is put in other vessel. Each vessel is warmed, and in case of preparing O/W type emulsion, the oil layer is gradually added to the aqueous layer, and in case of preparing W/O type emulsion, in contrast, the aqueous layer is gradually added to the oil layer. Each mixture continues to be mixed until the emulsifying is completed.

In case of preparing the aerosols, an ointment, a cream, a gel, a suspension, an emulsion, a solution or a lotion, etc. is prepared by the method as mentioned above and then, it is poured with a liquid gas or a compressed gas in a sealed vessel to prepare the aerosols.

The diseases which the external preparation of the present invention is applied to are, as mentined above, xerosis cutis, with itching and dermatosis, such as injured skin, eczema, rubor, ulticaria, pruritus, bulla, edema, etc. in patients receiving hemodialysis treatment.

### MODE FOR CARRYING OUT THE INVENTION

The external preparations containing acetylsalicylic acid of the present invention are explained by examples and experimental examples, but the present invention is not limited by these examples.

### EXAMPLES 1-6

### Ointments

According to ingredients indicated in Table 1, an ointment base (vaseline or hydrocarbon gel), a solvent (Tween 80, crotamiton, an alcohol, diisopropyl adipate or isopropyl myristate) and Aspirin were dissolved or dispersed under well stirring on a water bath. Then the mixture was cooled under stirring to prepare ointments.

TABLE 1
Ingredients of ointments containing Aspirin

| | Examples | | | | | |
|---|---|---|---|---|---|---|
| | 1 | 2 | 3 | 4 | 5 | 6 |
| Ingredients | Ingredient ratio (wt %) | | | | | |
| Aspirin | 0.1 | 1.0 | 10.0 | 0.5 | 5.0 | 2.0 |
| Ethanol | 1.0 | 2.0 | 10.0 | — | — | — |
| Tween 80 | — | — | — | 5.0 | — | — |
| Crotamiton | — | — | — | — | 5.0 | — |
| Diisopropyl adipate | — | — | — | — | — | 5.0 |
| Vaseline | 98.9 | — | 80.0 | 94.5 | — | — |
| Hydrocarbon gel | — | 97.0 | — | — | 90.0 | 97.0 |

### EXAMPLES 7-9

### Lotions

According to ingredients indicated in Table 2, Aspirin was added to a warmed oil layer to be dissolved or dispersed. Separately other ingredients were dissolved in previously warmed purified water, and the oil layer was added thereto under vigorously stirring. The mixture was mixed to homogeneity under gradually cooling to prepare lotions.

TABLE 2
Ingredients of lotions containing Aspirin

| | Examples | | |
|---|---|---|---|
| | 7 | 8 | 9 |
| Ingredients | Ingredient ratio (wt %) | | |
| Aspirin | 0.05 | 0.5 | 5.0 |
| Crotamiton | 1.0 | 2.0 | 5.0 |
| Isopropanol | — | — | 2.0 |
| Diisopropyl sebacate | 1.0 | — | — |
| Squalane | 3.0 | 3.0 | 3.0 |
| Cetanol | 3.0 | 3.0 | 3.0 |
| Solbitan sesquioleate | 0.5 | 0.5 | 0.5 |
| Polyoxy(20)cetyl ether | 1.5 | 1.5 | 1.5 |
| Propylene glycol | 5.0 | 5.0 | 5.0 |
| Triethanolamine | 0.4 | 0.4 | 0.4 |
| Purified water | 84.55 | 77.0 | 74.6 |

### EXAMPLES 10-12

### Gels

According to ingredients indicated in Table 3, after a water soluble polymer was melted on a water bath, Aspirin was dissolved or dispersed in a solvent(s) and these ingredients with other bases were homogeneously mixed to prepare gels.

TABLE 3
Ingredients of gels containing Aspirin

| | Examples | | |
|---|---|---|---|
| | 10 | 11 | 12 |
| Ingredients | Ingredient ratio (wt %) | | |
| Aspirin | 0.2 | 2.0 | 20.0 |
| Crotamiton | 1.0 | 5.0 | 2.0 |
| Isopropanol | 1.0 | — | 2.0 |
| Propylene glycol | 45.0 | 45.0 | 36.0 |
| Polyacrylic acid | 25.0 | 25.0 | 25.0 |
| Triethanolamine | 0.7 | 0.7 | 0.7 |
| Purified water | 27.1 | 22.3 | 14.3 |

### EXAMPLES 13-15

### Creams

According to ingredients indicated in Table 4, after a solid base was melted on a water bath, Aspirin dissolved or dispersed in a solvent was added thereto. A water-soluble base was dissolved in water and its warmed solution was added to the mixture. The mixture was kneaded until it became homogenous to prepare creams.

TABLE 4
Ingredients of ointments containing Aspirin

| | Examples | | |
|---|---|---|---|
| | 13 | 14 | 15 |
| Ingredients | Ingredient ratio (wt %) | | |
| Aspirin | 4.0 | 4.0 | 4.0 |
| Crotamiton | 1.0 | 1.0 | 1.0 |
| Sesame oil | 2.0 | — | 1.0 |
| Diisopropyl sebacate | — | 2.0 | 1.0 |
| Cetanol | 9.0 | 9.0 | 9.0 |
| White vaseline | 8.0 | 8.0 | 8.0 |
| Hexyldecanol | 1.0 | 1.0 | 1.0 |
| Polyethylene glycol monostearate | 2.0 | 2.0 | 2.0 |
| Polyoxy(9)lauryl ether | 2.8 | 2.8 | 2.8 |
| Polyoxy(23)cetyl ether | 2.0 | 2.0 | 2.0 |
| Propylene glycol | 12.0 | 12.0 | 12.0 |
| Methylparaben | 0.1 | 0.1 | 0.1 |
| Propylparaben | 0.1 | 0.1 | 0.1 |
| Purified water | 54.0 | 54.0 | 54.0 |

### EXAMPLES 16-18

### Tapes

According to ingredients indicated in Table 5, to a tacking agent consisting of an acrylate resin or a stylene-isoprene-stylene block copolymer were added an alicyclic saturated hydrocarbon resin, liquid paraffin, polybutene, an antioxidant, etc. and the mixture was dissolved in an organic solvent such as toluene etc. under stirring, or the mixture was melted by heating under stirring. Thereto was added Aspirin and the resulting mixture was spread on release paper and then in case of a soluble type, was spread on release paper and dried. The release paper was laminated on a flexible support to be cut into a desired size to prepare tapes.

TABLE 5
Ingredients of tapes containing Aspirin

| | Examples | | |
|---|---|---|---|
| | 16 | 17 | 18 |
| Ingredients | Ingredient ratio (wt %) | | |
| Aspirin | 5.0 | 10.0 | 30.0 |
| Isopropyl myristate | 5.0 | — | — |
| Diisopropyl adipate | — | — | 5.0 |
| Crotamiton | 5.0 | 5.0 | 1.0 |
| Acrylate-vinyl acetate copolymer | — | 85.0 | — |
| Stylene-isoprene-stylene | 20.0 | — | 13.4 |
| block copolymer | | | |
| Alicyclic saturated | 42.0 | — | 23.5 |
| hydrocarbon resin | | | |
| Polybutene | 15.0 | — | 11.6 |
| Liquid paraffin | 7.0 | — | 14.5 |
| Dibutyl hydroxytoluene | 1.0 | — | 1.0 |

### EXAMPLES 19-21

### Powders

According to ingredients indicated in Table 6, potato starch, zinc oxide and Aspirin were well mixed to prepare powders.

TABLE 6
Ingredients of powders containing Aspirin

| | Examples | | |
|---|---|---|---|
| | 19 | 20 | 21 |
| Ingredients | Ingredient ratio (wt %) | | |
| Aspirin | 20.0 | 40.0 | 80.0 |
| Potato starch | 76.0 | 56.0 | 16.0 |
| Zinc oxide | 4.0 | 4.0 | 4.0 |

### COMPARATIVE EXAMPLES 1-3

According to the method of preparing ointments, ointments having an ingredient(s) of Table 7 (comparative examples 1-3) were prepared.

TABLE 7
Ingredients of ointments (Comparative examples)

| | Comparative examples | | |
|---|---|---|---|
| | 1 | 2 | 3 |
| Ingredients | Ingredient ratio (wt %) | | |
| Diphenhydramine | 1.0 | — | |
| Dexamethasone | — | 0.1 | |
| Propylene glycol | 10.0 | 10.0 | |
| Isopropyl myristate | 5.0 | 5.0 | |
| Hydrocarbon gel | 84.0 | 84.9 | 100.0 |

Clinical Tests:

An antipruritic activity was tested by administering the external preparations of the present invention and control-preparations to patients receiving hemodialysis (volunteers).

The degree of improvement of skin condition or itching was evaluated based on the following five steps standard:

A: Remarkably effective,

B: Effective,

C: Slightly effective,

D: No change,

E: Worse.

Being slightly effective (C) or more than slightly effective (A, B), degree was judged to be effective, and its effective rate was calculated.

Experiment 1: Improvement of Skin Conditions

The external preparation of the present invention containing Aspirin was administered to affected lesions of 10 patients having dermatosis (bad skin conditions) and skin itching among patients receiving hemodialysis treatment. Degree of improvement of the skin conditions was evaluated.

As controls, the preparations of comparative examples 1 to 3 were administered to the 7 patients having dermatosis and skin itching in the same manner as above.

The result was shown in Table 8.

TABLE 8
Degree of improvement of skin conditions due to
hemodialysis

| | Drugs | No. of | Evaluation | | | | | Effective |
|---|---|---|---|---|---|---|---|---|
| Groups | (wt %) | patient | A | B | C | D | E | rate(%) |
| Example 2 | Aspirin | 2 | 1 | 0 | 1 | 0 | 0 | 100.0 |
| | (1.0) | | | | | | | |
| Example 4 | Aspirin | 3 | 2 | 1 | 0 | 0 | 0 | 100.0 |
| | (0.5) | | | | | | | |
| Example 6 | Aspirin | 3 | 3 | 0 | 0 | 0 | 0 | 100.0 |
| | (2.0) | | | | | | | |
| Example 13 | Aspirin | 2 | 0 | 1 | 0 | 1 | 0 | 50.0 |
| | (4.0) | | | | | | | |
| Comp. | Diphen- | 3 | 0 | 0 | 0 | 3 | 0 | 0 |
| Ex. 1 | hydramine | | | | | | | |
| | (1.0) | | | | | | | |
| Comp. | Dexa- | 3 | 0 | 0 | 1 | 2 | 0 | 33.0 |
| Ex. 2 | methasone | | | | | | | |
| | (0.1) | | | | | | | |
| Comp. | — | 1 | 0 | 0 | 0 | 0 | 1 | 0 |
| Ex. 3 | | | | | | | | |

As is clear from the result in Table 8, preparations of examples 2, 4, 6 and 13 containing Aspirin (the preparations of the present invention) showed improvement of the skin condition due to hemodialysis and the effects were strong, comparing with controls (preparations of comparative examples 1 to 3).

Experiment 2: Improvement of Itching

The external preparation containing Aspirin was administered to affected lesions of 12 patients having itching among patients receiving hemodialysis treatment. Degree of improvement of the itching was evaluated.

As controls, the preparations of comparative example 1 to 3 were administered to the 7 patients having itching in the same manner as above.

The result was shown in Table 9.

TABLE 9
Degree of improvement of itching due to
hemodialysis

| | Drugs | No. of | Evaluation | | | | | Effective |
|---|---|---|---|---|---|---|---|---|
| Groups | (wt %) | patient | A | B | C | D | E | rate(%) |
| Example 2 | Aspirin | 2 | 1 | 1 | 0 | 0 | 0 | 100.0 |
| | (1.0) | | | | | | | |
| Example 4 | Aspirin | 3 | 2 | 1 | 0 | 0 | 0 | 100.0 |
| | (0.5) | | | | | | | |
| Example 6 | Aspirin | 3 | 2 | 0 | 1 | 0 | 0 | 100.0 |
| | (2.0) | | | | | | | |
| Example 13 | Aspirin | 2 | 1 | 1 | 0 | 0 | 0 | 100.0 |
| | (4.0) | | | | | | | |
| Example 17 | Aspirin | 2 | 0 | 1 | 1 | 0 | 0 | 100.0 |
| | (10.0) | | | | | | | |
| Comp. | Diphen- | 3 | 0 | 0 | 2 | 1 | 0 | 0 |
| Ex.1 | hydramine | | | | | | | |
| | (1.0) | | | | | | | |
| Comp. | Dexa- | 3 | 0 | 0 | 1 | 2 | 0 | 33.0 |
| Ex.2 | methasone | | | | | | | |
| | (0.1) | | | | | | | |
| Comp. | — | 1 | 0 | 0 | 0 | 0 | 1 | 0 |
| Ex. 3 | | | | | | | | |

As is clear from the result in Table 9, preparations of examples 2, 4, 6, 13 and 17 containing Aspirin (the preparations of the present invention) inhibited the itching due to hemodialysis and showed very strong antipruritic activity, comparing with controls (the preparations of comparative examples 1 to 3).

### POSSIBILITY OF INDUSTRIAL APPLICABILITY

The external preparation of the present invention containing Aspirin as an active ingredient has an excellent therapeutic effect to itching due to hemodialysis. Furthermore, the external preparation of the present invention shows improvement for skin conditions as well as sedative for itching. The external preparation of the present invention does not affect any value of clinical assay (pathology) and belongs to the drug showing very little side effects and therefore, is considered as a safety medicament. The present invention can provide the external preparation being not only excellently effective to various itching due to hemodialysis, but also shows improvement for skin conditions and being very little in its side effects.

1. A method for treating dermotosis or pruritus due to hemodialysis which comprises applying to an affected lesion of a patient an external preparation containing acetylsalicylic acid as an active ingredient.

2. The method according to claim 1 wherein the disease treated is xerosis cutis or itching.

3. Use of acetylsalicylic acid for the external preparation for treating dermotosis or pruritus due to hemodialysis.

4. The use according to claim 3 wherein the disease treated is xerosis cutis or itching.

5. An external composition containing acetylsalicylic acid as an active ingredient for treating dermotosis or pruritus due to hemodialysis.

6. The composition according to claim 5 wherein the disease treated is xerosis cutis or itching.
